(19) United States
(12) Patent Application Publication
(10) Pub. No.: US 2023/0070934 A1
(43) Pub. Date: Mar. 9, 2023

(54) ARTICLE AND METHOD TO TREAT INTERPROXIMAL DENTAL CARIES

(71) Applicant: 3M INNOVATIVE PROPERTIES COMPANY, St. Paul, MN (US)

(72) Inventors: Yizhong WANG, Woodbury, MN (US); Jamie VADNAIS, Cottage Grove, MN (US); Tiffany T. TON, Woodbury, MN (US); Richard P. RUSIN, Woodbury, MN (US)

(21) Appl. No.: 17/759,406

(22) PCT Filed: Jan. 28, 2021
(86) PCT No.: PCT/IB2021/050678

§ 371 Date: Jul. 25, 2022

Related U.S. Application Data
(60) Provisional application No. 62/968,104, filed on Jan. 30, 2020.

(51) Int. Cl. A61Q 11/00; A61K 8/21

(52) U.S. Cl. CPC A61Q 11/00; A61K 8/21

(57) ABSTRACT

Silver-fluoride compositions, dental articles comprising silver-fluoride compositions, and methods for reducing bacteria on a tooth surface and/or remineralizing a tooth surface are described.

### BACKGROUND

Dental caries is a disease in which tooth decay results from interaction with acid produced by bacteria. Silver salts are known to have antibacterial properties and fluoride salts are known to remineralize tooth surfaces. Both silver and fluoride compositions have separately been shown to aid in preventing and arresting caries activities.

Silver diamine fluoride has been used to treat surfaces affected by caries; however, reaction with saliva causes the formation of silver phosphate. Silver phosphate is light sensitive and permanently stains teeth black when exposed to light. Silver fluoride (without an amine or other ligand stabilizer) is extremely unstable in aqueous solutions and quickly decomposes to form metallic silver.

It was has recently demonstrated in Int. Pat. App. No. IB2019/056376, the contents of which are incorporated herein by reference upon publication, that a stable form of aqueous silver cations and fluoride anions is achievable for treating caries or preventing caries progression.

What is needed is a way to treat a tooth surface with silver and fluoride without requiring an arduous treatment plan and mitigating fluoride ingestion. In particular, there is a need to treat interproximal tooth surfaces in an efficient, safe, and aesthetically-pleasing manner.

### SUMMARY

In one embodiment, a dental article configured to access an interproximal gap is described. The dental article may include at least one applicator, a silver-fluoride composition, and a package to contain one or more of the at least one applicator and the silver fluoride composition. The silver-fluoride composition may include a source of silver cations, a source of fluoride anions, a source of iodide or thiocyanate anions, and water. The package may be impermeable to water.

In one embodiment, a dental article is described. The dental article may include at least one applicator, a silver-fluoride composition, and a package to contain one or more of the at least one applicator and the silver fluoride composition. The silver-fluoride composition may include a source of silver cations, a source of fluoride anions, a source of iodide or thiocyanate anions, and water. The package may be impermeable to water.

In one embodiment, a method for one or more of reducing bacteria on a tooth surface and remineralizing a tooth surface is described. The method may include providing at least one applicator and a silver-fluoride composition, contacting an effective amount of the silver-fluoride composition to the tooth surface with the at least one applicator, and allowing the silver-fluoride composition to contact the tooth surface for a period. The silver-fluoride composition may include a source of silver cations, a source of fluoride anions, a source of iodide or thiocyanate anions, and water. The silver-fluoride composition may one or more of reduce bacteria at the tooth surface and remineralize a tooth surface.

In one embodiment, a method for reducing bacteria on a tooth surface is described. The method may include providing at least one applicator and a silver-fluoride composition, contacting an effective amount of the silver-fluoride composition to the tooth surface with the at least one applicator, and allowing the silver-fluoride composition to contact the tooth surface for a period. The silver-fluoride composition may include a source of silver cations, a source of fluoride anions, a source of iodide or thiocyanate anions, and water. The silver-fluoride composition may reduce bacteria at the tooth surface.

In one embodiment, a method for remineralizing a tooth surface is described. The method may include providing at least one applicator and a silver-fluoride composition, contacting an effective amount of the silver-fluoride composition to the tooth surface with the at least one applicator, and allowing the silver-fluoride composition to contact the tooth surface for a period. The silver-fluoride composition may include a source of silver cations, a source of fluoride anions, a source of iodide or thiocyanate anions, and water. The silver-fluoride composition may remineralize the tooth surface.

In one embodiment, a kit is provided. The kit may include a dental article including a at least one applicator and a silver-fluoride composition. Alternatively, the kit may include at least one applicator and a silver-fluoride composition. The silver fluoride composition(s) may include a source of silver cations, a source of fluoride anions, a source of iodide or thiocyanate anions, and water. The kit may further include a set of instructions direct a user perform method steps for reducing bacteria at a tooth surface described herein or methods steps for remineralizing a tooth surface described herein.

### BRIEF DESCRIPTION OF DRAWINGS

FIG. 1A is an example applicator of the present disclosure.

FIG. 1B is an example applicator of the present disclosure.

FIG. 1C is an example applicator of the present disclosure.

FIG. 2 is an example package of the present disclosure.

FIG. 3A is an example dental article the present disclosure.

FIG. 3B is an example dental article of the present disclosure.

FIG. 4A is an example dental article of the present disclosure.

FIG. 4B is an example dental article of the present disclosure.

FIG. 5A is an example dental article of the present disclosure.

FIG. 5B is an example dental article of the present disclosure.

FIG. 5C is an example dental article of the present disclosure.

FIG. 6 is a flowchart illustrating a method of the present disclosure.

FIG. 7A is an example kit of the present disclosure.

FIG. 7B is an example kit of the present disclosure.

### DETAILED DESCRIPTION

The silver-fluoride compositions described herein are effective in arresting caries activity and repairing the damage caused by caries bacteria without staining teeth. However, the silver-fluoride composition requires water to remain effective. In the absence of sufficient water, the silver-fluoride composition will decompose and may cause discoloration of teeth. The dental articles described herein provide a way to extend the shelf-life of the silver-fluoride composition (at a calculated estimate of about 2 years) by preventing water evaporation. Furthermore, the dental articles described herein provide a means to reach interproximal areas and areas below the gumline that may be affected by caries without having to use the composition in excess.

As used herein, “about” means±10 percent of a given value. For example, about 10 means 9 to 11.

As used herein, “silver-fluoride composition” refers to a stable aqueous solution having complex formed from silver and fluoride ions. Without wishing to be bound by theory, it is believed that the silver-fluoride composition includes a stable Ag(I) fluoride complex that is stabilized by iodine or thiocyanate and water.

As used herein, “impermeable” is used to define a material that does not allow for the passage of water or water vapor in any appreciable amount, i.e., The Moisture Vapor Transmission Rate (MVTR) of the material should be less than 0.0005 gram per 100 square inches in 24 hrs. The impermeable packages described herein protects the silver-fluoride composition from decomposition due to loss of water.

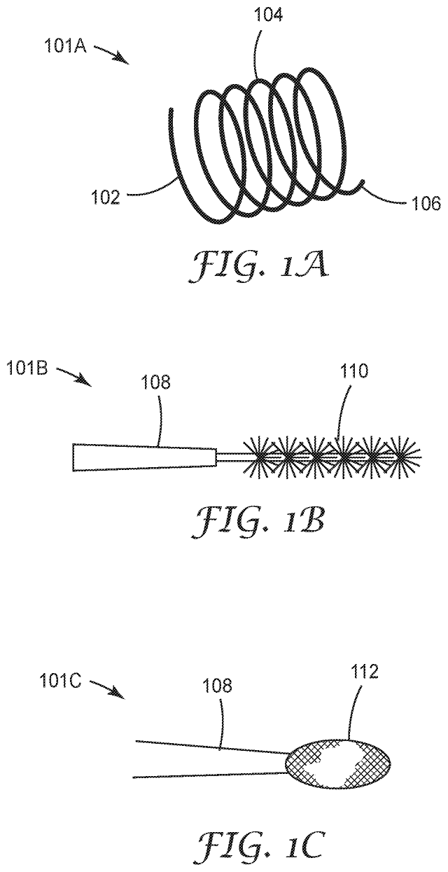

FIG. 1A illustrates an example floss applicator 101A. Floss applicator 101A may include a standard floss portion 102, a spongy portion 104, and a threading portion 106.

FIG. 1B illustrates an example microbrush applicator 101B. Microbrush applicator 101B may include a handle 108B and a brush tip 110.

FIG. 1C illustrates an example swab applicator 101C. Swab applicator 101C may include a handle 108C and a swab tip 112.

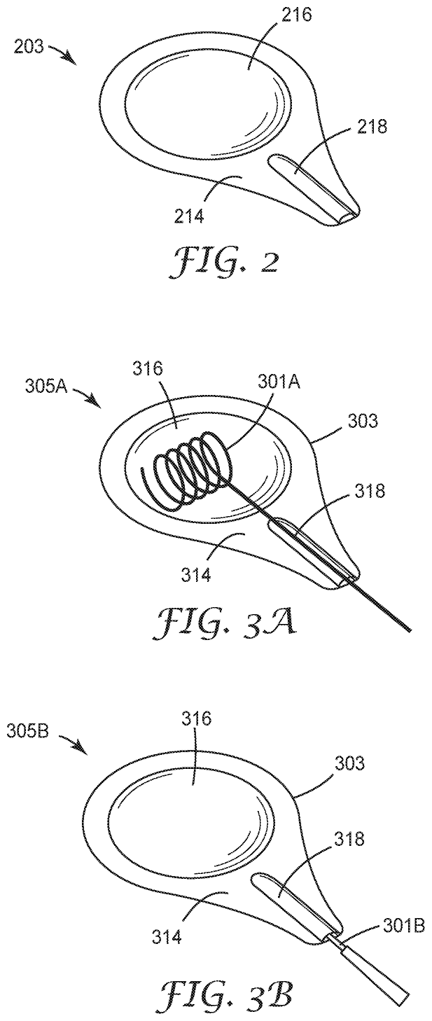

FIG. 2 illustrates an example blister-type package 203. Blister-type package 203 may include a sealed perimeter body 214 having a cavity 216 for storing a silver-fluoride composition. Blister-type package 203 may further include an exit path 218 for expelling the silver-fluoride composition.

FIG. 3A illustrates an example dental article 305A having a blister-type package 303. Blister-type package 303 may include a sealed perimeter body 314 having a cavity 316 for storing a silver-fluoride composition. Blister-type package 303 may further include an exit port 318 for expelling the silver-fluoride composition. Dental article 305A is further shown to house a floss applicator 301A within cavity 316 and that floss applicator 301A may be removed from cavity 316 via exit port 318.

FIG. 3B illustrates an example dental article 305B having a blister-type package 303. Blister-type package 303 may include a sealed perimeter body 314 having a cavity 316 for storing a silver-fluoride composition. Blister-type package 303 may further include an exit port 318 for expelling the silver-fluoride composition. Dental article 305B is further shown to house a microbrush applicator 301A (or swab not shown) within exit port 318.

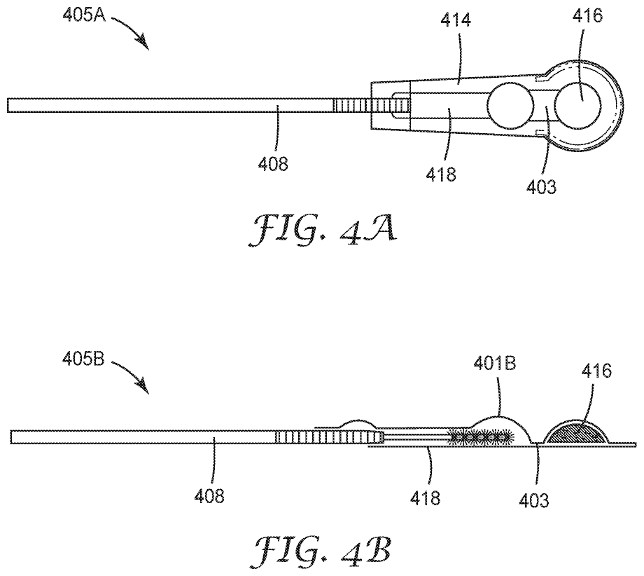

FIG. 4A illustrates an example dental article 405A. Dental article 405A may include a blister-type package 403. Blister-type package 403 may include a sealed perimeter body 414 having a cavity 416 for storing a silver-fluoride composition. Blister-type package 403 may further include an exit path 418 for expelling the silver-fluoride composition. Dental article 405A further shows a handle 408 of a microbrush or swab applicator.

FIG. 4A illustrates an example dental article 405A. Dental article 405A may include a blister-type package 403. Blister-type package 403 may include a sealed perimeter body 414 having a cavity 416 for storing a silver-fluoride composition. Blister-type package 403 may further include an exit path 418 for expelling the silver-fluoride composition. Dental article 405A further shows a handle 408 of a microbrush applicator 410B.

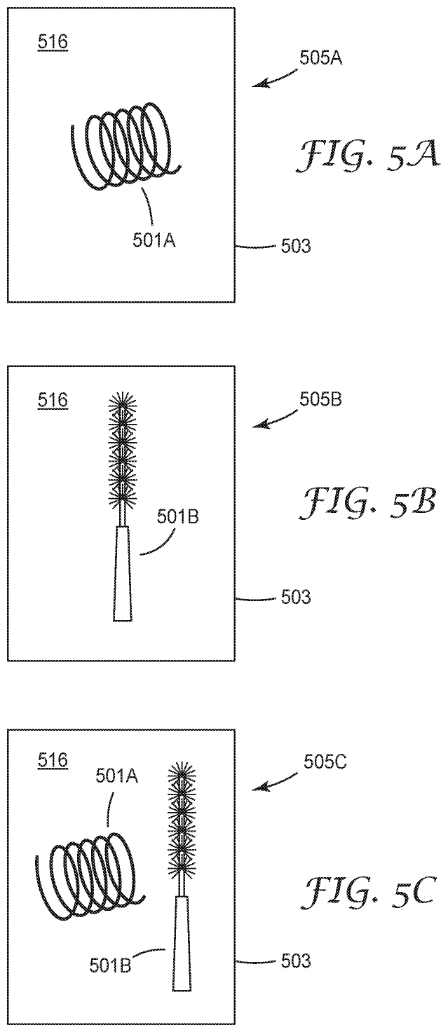

FIG. 5A illustrates an example dental article 505A. Dental article 505A may include a foil pouch-type package 503. Foil pouch-type package 503 may include a cavity 516 for housing a floss applicator 501A (or swab not shown) and the silver-fluoride composition.

FIG. 5B illustrates an example dental article 505B. Dental article 505B may include a foil pouch-type package 503. Foil pouch-type package 503 may include a cavity 516 for housing a microbrush applicator 501B (or swab not shown) and the silver-fluoride composition.

FIG. 5C illustrates an example dental article 505C. Dental article 505C may include a foil pouch-type package 503. Foil pouch-type package 503 may include a cavity 516 for housing a floss applicator 501A and a microbrush applicator 501B (or swab not shown) and the silver-fluoride composition.

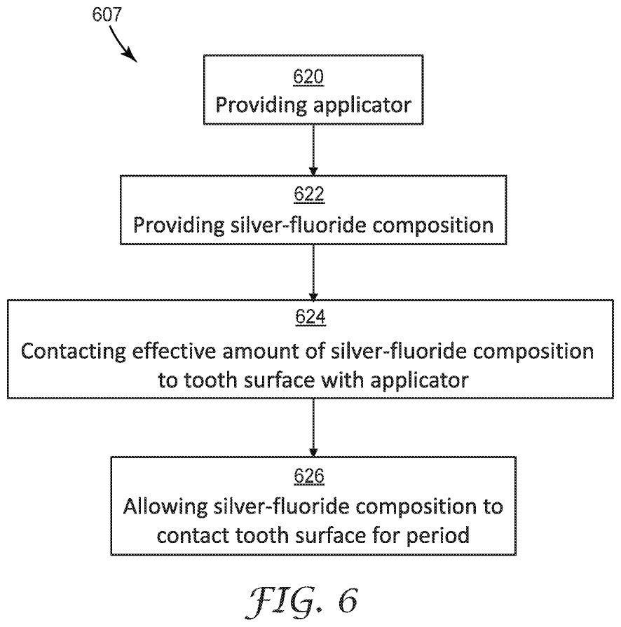

FIG. 6 illustrates a method 607 of the present disclosure. Method 607 may include 620 providing an applicator, 622 providing a silver-fluoride composition, 624 contacting an effective amount of the silver-fluoride composition to a tooth surface with the applicator, and 626 allowing the silver-fluoride composition to contact the tooth surface for a period.

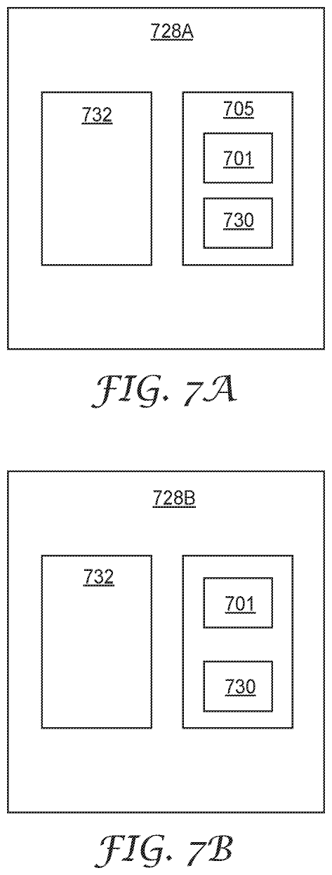

FIG. 7A illustrates an example kit 728A. Kit 728A may include a dental article 705. Dental article 705 may include an applicator 701 and a silver-fluoride composition 730. Kit 728A may further include a set of instructions 732 directing a user to perform the steps of a method, e.g., method 607, described herein.

FIG. 7B illustrates an example kit 728B. Kit 728B may include an applicator 701 and a silver-fluoride composition 730. Kit 728B may further include a set of instructions 732 directing a user to perform the steps of a method, e.g., method 607, described herein.

### Dental Articles

In several embodiments, a dental article or a dental article configured to access an interproximal gap is described. The dental article may include at least one applicator, a silver-fluoride composition, and a package to contain one or more of the at least one applicator and the silver fluoride composition. The silver-fluoride composition may include a source of silver cations, a source of fluoride anions, a source of iodide or thiocyanate anions, and water. The package may be impermeable to water.

In many embodiments, the dental article includes one or more applicator described herein.

In some embodiments, at least one applicator may be loaded with the silver-fluoride composition within the package. For example, the applicator may be stored in a solution of the silver-fluoride composition. In another example, the silver-fluoride composition may be absorbed on or within the applicator.

In other embodiments, the at least one applicator may be separated from the silver-fluoride composition within the package.

In other embodiments, the at least one applicator may be loaded with the silver-fluoride composition within the package, and the package may further include a separate compartment the silver-fluoride composition.

In some embodiments, the dental article may include two applicators described herein.

In some embodiments, one or more applicator may be loaded with the silver-fluoride composition within the package. In some embodiments one applicator is loaded with the silver-fluoride composition in the package.

In some embodiments, the silver-fluoride composition may include silver cations present in an amount of about 13-20 wt % with respect to the weight of the silver-fluoride composition. For example, the silver cations may be present in an amount in wt % with respect to the weight of the silver-fluoride composition of about 13.0, 13.25, 13.5, 13.75, 14.0, 14.25, 14.5, 14.75, 15.0, 15.25, 15.5, 15.75, 16.0, 16.25, 16.5, 16.75, or 17.0, 17.5, 18.0, 18.5, 19.0, 19.5, or 20.0, or a value within a range between any of the preceding values, for example, between about 13.5 and about 15.0, between about 14.0 and about 16.75, between about 13.0 to about 17.0, or the like. The source of silver anions may be any source described herein.

In some embodiments, the silver-fluoride composition may include fluoride anions present in an amount of about 2.0-4.0 wt % with respect to the weight of the silver-fluoride composition. For example, the silver cations may be present in an amount in wt % with respect to the weight of the silver-fluoride composition of about 2.0, 2.25, 2.30, 2.35, 2.40, 2.45, 2.50, 2.55, 2.60, 2.65, 2.70, 2.75, 2.80, 2.85, 2.90, 3.0, 3.1, 3.2, 3.3, 3.4, 3.5, 3.6, 3.7, 3.8, 3.9, or 4.0, or a value within a range between any of the preceding values, for example, between about 2.50 and about 2.70, between about 2.35 and about 2.85, or the like. The source of fluoride anions may be any source described herein. In some embodiments, the silver-fluoride composition may include fluoride anions present in an amount of about 2.0-3.6 wt % with respect to the weight of the silver-fluoride composition.

In some embodiments, the silver-fluoride composition may include silver cations and iodide anions present in a molar ratio of less than about 0.42:1 respectively. For example, silver cations may be present in a molar ratio relative to iodide anions of about 0.42:1, 0.40:1, 0.38:1, 0.36:1, 0.34:1, 0.32:1, 0.30:1, 0.28:1, 0.26:1, 0.24:1, 0.22:1, 0.20:1, 0.18:1, 0.16:1, 0.14:1, 0.12:1, or 0.10:1, or a value within a range between any of the preceding values, for example, between about 0.35:1 and about 0.40:1, between about 0.22:1 and about 0.32:1, or the like. In some embodiments, the silver cations and iodide anions may be present in a molar ratio of less than about 0.10:1. The source of iodide anions may be any source described herein.

In some embodiments, the silver-fluoride composition may include silver cations and thiocyanate anions present in a molar ratio of less than about 0.37:1. In some embodiments, the silver-fluoride composition may include silver cations and thiocyanate anions present in a molar ratio of between about 0.1:1 and about 0.37:1. For example, silver cations may be present in a molar ratio relative to thiocyanate anions of about 0.1:1, 0.15:1, 0.2:1, 0.25:1, 0.30:1, 0.35:1 or 0.37:1, or a value within a range between any of the preceding values, for example, between about 0.15:1 to about 0.25:1, between about 0.1:1 and about 0.2:1, or the like. The source of thiocyanate anions may be any source described herein.

In some embodiments, the silver-fluoride composition may include water present in an amount less than about 45 wt % with respect to the weight of the silver-fluoride composition. For example, water may be present in an amount in wt % with respect to the weight of the silver-fluoride composition of about 45, 44, 43, 42, 41, 40, 35, 30, 25, 20, 15, and 10, or a value within a range between any of the preceding values, for example, between about 40 and about 44, between about 30 and about 35, between about 20 and about 45, or the like. In some embodiments, the silver-fluoride composition includes at least 20 wt % to maintain the structural integrity of the silver complex.

In some embodiments, the silver-fluoride composition having iodide anions may include water present in an amount less than 41.2 wt-% with respect to the weight of the silver-fluoride composition.

In some embodiments, the silver-fluoride composition having thiocyanate anions may include water present in an amount less than 57.1 wt-% with respect to the weight of the silver-fluoride composition.

In many embodiments, the silver-fluoride compositions described herein are stable to light. In other words, the silver-fluoride compositions are not sensitive to light and thereby do not decompose into species that cause blackening of teeth. In some embodiments, the silver-fluoride compositions are stable to LED curing light at a wavelength of 450 nm.

### Applicator

In many embodiments, any applicator described herein may include a means for transferring a silver-fluoride composition to a tooth surface. In some embodiments, an applicator may have an absorbent or adsorbent surface. The absorbent or adsorbent surface may include hydrophilic components. In other embodiments, an applicator may include an area configured to hold a solution via capillary action.

In some embodiments, an applicator may be selected from a floss, a microbrush, a pick, a pick including a floss (floss pick), a swab, and a combination thereof, or the like.

In some embodiments, an applicator may be selected from a floss and a floss pick.

In some embodiments, an applicator may be selected from a floss, a floss pick, and a microbrush.

In some embodiments, an applicator may be selected from a floss and a microbrush.

In some embodiments, an applicator may be a floss. The floss may be one or more of tangled, twisted, and braided.

In some embodiments, an applicator may be or include a floss. For example, a floss may include any floss known in the art, such as those made from nylon, polyester, or the like. In some embodiments, the floss may be one or more of tangled, twisted, and braided. The floss may include a coating conducive to absorbing or adsorbing an aqueous composition. In some embodiments, the floss may include a threader, e.g., a stiffened-end, a spongy floss portion, and a standard floss portion, such as used in the floss sold under the tradename Super Floss® (Oral-B; Iowa City, Iowa). The spongy floss portion may include one or more of a diameter thicker than the standard floss portion and material having a transferring capacity, e.g., absorbent, adsorbent, capillary action properties, or the like.

In some embodiments, an applicator may include a pick including a floss. The pick may include a set of prongs having a floss extending between. The floss may include any floss known in art and described herein.

In some embodiments, an applicator may be or include a microbrush. The microbrush may include fibers, bristles, or the like.

In some embodiments, more than one applicator may be used to the deliver the silver-fluoride composition. For example, a floss may be loaded with a microbrush having a silver-fluoride composition disposed thereon, and the floss contacted to a tooth surface. For example, a floss may be contacted to a tooth surface and a microbrush having a silver-fluoride composition disposed thereon may load the floss in contact with the tooth surface.

### Source of Silver Cations

In some embodiments, the source of silver cations may be selected from one or more of silver fluoride, silver chloride, silver nitrate, silver iodide, and silver diamine fluoride.

In some embodiments, the source of silver cations may be selected from one or more of silver fluoride, silver chloride, silver nitrate, and silver iodide.

In some embodiments, the source of silver cations is silver iodide.

### Source of Fluoride Anions

In some embodiments, the source of fluoride anions is selected from one or more of silver fluoride, silver diamine fluoride, sodium fluoride, ammonium fluoride, potassium fluoride, and an organic ammonium fluoride.

As used herein, “organic ammonium fluoride” describes a compound of the formula R₄N⁺R⁻, wherein at least one R is a C₄-C₂₆ hydrocarbon. Example organic ammonium fluorides include 3-[octadecyl(2-hydroxyethyl)amino]propyl]bis(2-hydroxyethyl)amine dihydrofluoride (OLAFLUR), CH₃(CH₂)₇CH═CH(CH₂)₈NH₃⁺F⁻ (DECTAFLUR), tetrabutyl ammonium fluoride, or the like.

In some embodiments, the source of fluoride anions is selected from one or more of silver fluoride, sodium fluoride, ammonium fluoride, potassium fluoride, and an organic ammonium fluoride.

In some embodiments, the source of fluoride anions is selected from one or more of silver fluoride, silver diamine fluoride, ammonium fluoride, potassium fluoride, and an organic ammonium fluoride.

In some embodiments, the source of fluoride anions is selected from one or more of silver fluoride, ammonium fluoride, potassium fluoride, and an organic ammonium fluoride.

In some embodiments, the source of fluoride anions is ammonium fluoride.

### Source of Iodide Anions

In some embodiments, the source of iodide anions is selected from one or more of ammonium iodide, sodium iodide, potassium iodide, and silver iodide.

In some embodiments, the source of iodide anions is selected from one or more of ammonium iodide, sodium iodide, and potassium iodide.

In some embodiments, the source of iodide anions is ammonium iodide and silver iodide.

In some embodiments, the source of iodide anions is ammonium iodide.

### Source of Thiocyanate Anions

In some embodiments, the source of thiocyanate anions is selected from one or more of ammonium thiocyanate, sodium thiocyanate, guanidinium thiocyanate, potassium thiocyanate, and silver thiocyanate.

In some embodiments, the source of thiocyanate anions is ammonium thiocyanate.

### Package

In many embodiments, the package may include any package known in the art that is impermeable to water on the order of MVTR less than 0.0005 gram per 100 square inches in 24 hrs., MVTR less than 0.005 gram per 100 square inches in 24 hrs.

In many embodiments, the package may include a metal foil. The metal foil may include metal components selected from aluminum, copper, chromium, gold, iron, manganese, molybdenum, nickel, niobium, silver, tin, titanium, tungsten, vanadium, zinc, and a combination (alloy) thereof. In some embodiments, the metal foil may include foil packaging such as those sold by Oliver™ Healthcare Packaging, e.g, Ofoil™ 48, dispos-a-vent®, or the like.

In many embodiments, the package may include an adhesive for package sealing. The adhesive may include any heat sealant known in the art. For example, the adhesive may include polyethylene terephthalate, polyethylene, or the like.

In some embodiments, the package may be a heat-sealed foil package having a silver-fluoride composition.

In some embodiments, the package may include a single-unit dosage of the silver-fluoride composition.

In other embodiments, the package may include multiple-unit dosages of the silver-fluoride composition.

In some embodiments, the package may be a heat-sealed foil package having a silver-fluoride composition. In some embodiments, the package may further include a floss or floss pick wherein the floss or floss pick may be stored in a compartment with the silver-fluoride composition, or in a compartment separate from the silver-fluoride composition. In some embodiments, the package may further include a microbrush wherein the microbrush may be stored in a compartment with the silver-fluoride composition, or in a compartment separate from the silver-fluoride composition. In some embodiments, the package may include a floss or floss pick and a microbrush, wherein the floss or floss pick and the microbrush may be independently stored in a compartment with the silver-fluoride composition, or in a compartment separate from the silver-fluoride composition.

In some embodiments, the package may be a blister pack, such as those resembling L-Pop™ sold by 3M ESPE (Maplewood, Minn.). In some embodiments, the blister pack may further include a microbrush. In other embodiments, the blister pack may further include a floss within the blister pack.

In some embodiments, the package may include a one or more pieces of floss or floss picks. In other embodiments, the package may include a spool of floss for multiple-use.

In some embodiments, the package may be resealable or otherwise have an effective mechanism for preventing moisture loss after opening.

In one embodiment, the package may be a container including a spool of floss suspended in a silver-fluoride composition. The container may include a network that separates the silver-fluoride composition from a floss exit point such that water vapor may condense back into the container.

### Methods

In one embodiment, a method for reducing bacteria on a tooth surface and remineralizing the tooth surface is described. The method may include providing at least one applicator and a silver-fluoride composition, contacting an effective amount of the silver-fluoride composition to the tooth surface with the at least one applicator, and allowing the silver-fluoride composition to contact the tooth surface for a period. The silver-fluoride composition may include a source of silver cations, a source of fluoride anions, a source of iodide or thiocyanate anions, and water. The silver-fluoride composition may reduce bacteria at the tooth surface and remineralize the tooth surface.

In some embodiments, the methods described herein may include any dental article described herein.

In many embodiments, the methods described herein arrest caries activity and repair damage to tooth structure caused by caries activity.

In some embodiments, the method may further include inserting an applicator onto the tooth surface, e.g., into an interproximal gap, and loading the applicator with the silver-fluoride composition. In some embodiments, the applicator is a floss or floss pick.

In other embodiments, the method may further include inserting an applicator one the tooth surface, e.g., into an interproximal gap, wherein the applicator is pre-loaded with the silver-fluoride composition. In some embodiments, the applicator is a floss or floss pick.

In some embodiments, the method may further include re-loading the applicator, e.g., a floss or floss pick, by one or more of submerging the applicator in the silver-fluoride composition or applying additional silver-fluoride composition to the applicator with a second loaded applicator, e.g., a microbrush. In some embodiments, applying additional silver-fluoride composition to the applicator may occur while the applicator is in contact with the tooth surface.

In some embodiments, method may include contacting an effective amount of the silver fluoride composition to the tooth surface, wherein an effective amount is at least about 30 mg of the silver-fluoride composition. In some embodiments, an effective amount is about 30 mg to about 150 mg. For example, the effective amount of silver-fluoride composition in mg may be about 30, 40, 50, 60, 70, 80, 90, 100, 110, 120, 130, 140 or 150, of a value in a range between any of the preceding values, for example, between about 50 and about 80, between about 30 and about 100, or the like. The effective amount may be delivered in one or more applicator loadings.

In some embodiments, the method may further include allowing the silver-fluoride composition to contact the tooth surface for a period of about 30 to about 120 seconds. For example, the silver-fluoride composition may be allowed to contact the tooth surface for a period in seconds of about 30, 40, 50, 60, 70, 80, 90, 100, 110, and 120, or a value between a range of any of the preceding values, e.g., between about 60 and about 90, between about 40 and about 100, or the like. When the method may include one or more applicator loading, each loading may be independently allowed to contact the tooth surface for any period described above.

In some embodiments, the applicator may be removed from the tooth surface while the silver-fluoride composition is allowed to contact the tooth surface.

In some embodiments, the method may further include scrubbing the tooth surface during the period in which the silver-fluoride composition contacts the tooth surface. The scrubbing may be performed with the applicator or another device.

In some embodiments, the method may further include cleaning the tooth surface prior to contacting the silver-fluoride composition to the tooth surface. In some embodiments, the method may further include cleaning the tooth surface after allowing the silver-fluoride composition to contact the tooth surface for a period.

In some embodiments, the method may further include drying the tooth surface prior to contacting the silver-fluoride composition to the tooth surface. In some embodiments, the method may further include drying the tooth surface after allowing the silver-fluoride composition to contact the tooth surface for a period.

In some embodiments, the method may further include cleaning and drying the tooth surface prior to contacting the silver-fluoride composition to the tooth surface. In some embodiments, the method may further include cleaning and drying the tooth surface after allowing the silver-fluoride composition to contact the tooth surface for a period.

In some embodiments, the method may further include applying a varnish after the silver-fluoride composition is allowed to contact the tooth surface for period. The varnish may include, for example, a source of fluoride and a resin.

In some embodiments, the tooth surface contacted by the silver-fluoride composition of the methods described herein may be any tooth surface accessible by the at least one applicator. In some embodiments, the tooth surface is an interproximal tooth surface. In other embodiments, the tooth surface is an occlusal surface. In some embodiments, the tooth surface is a buccal surface. In some embodiments, the tooth surface is a lingual surface. In some embodiments, the tooth surface is below the gumline.

In many embodiments, the methods described herein may include any applicator(s) described herein.

In many embodiments, the methods described herein may include any silver-fluoride composition described herein, including any source of cations, anions, or the like described herein.

In many embodiments, the methods described herein may include any package(s) described herein.

### Reducing Bacteria

In one embodiment, a method for reducing bacteria on a tooth surface is described. The method may include providing at least one applicator and a silver-fluoride composition, contacting an effective amount of the silver-fluoride composition to the tooth surface with the at least one applicator, and allowing the silver-fluoride composition to contact the tooth surface for a period. The silver-fluoride composition may include a source of silver cations, a source of fluoride anions, a source of iodide or thiocyanate anions, and water. The silver-fluoride composition may reduce bacteria at the tooth surface.

In some embodiment, the method for reducing bacteria on a tooth surface may further include any additional steps or features described above.

In some embodiments, the reduction of bacteria may be quantified or qualified by log reduction or zone inhibition, respectively. For example, the methods described herein may provide a log reduction of caries-causing bacteria about 1 to 6. For example, the methods described herein may provide a zone inhibition of caries-causing bacteria of about 6 mm to about 20 mm.

In many embodiments, the methods described herein are especially suited for reducing bacteria on an interproximal tooth surface, i.e., between adjacent teeth.

### Remineralization

In many embodiments, a method for remineralizing a tooth surface is described. The method may include providing at least one applicator and a silver-fluoride composition, contacting an effective amount of the silver-fluoride composition to the tooth surface with the at least one applicator, and allowing the silver-fluoride composition to contact the tooth surface for a period. The silver-fluoride composition may include a source of silver cations, a source of fluoride anions, a source of iodide or thiocyanate anions, and water. The silver-fluoride composition may remineralize the tooth surface.

In some embodiment, the method for remineralizing a tooth surface may further include any additional steps or features described above.

In some embodiments, the remineralization of the tooth surface may be quantified by an increase in hardness as measured by indentation hardness measurements with an indentor, such as Vickers (VHN) or Knoop (KHN). For example, the methods described herein may provide an increase in hardness of about VHN 10 to about VHN 350.

In many embodiments, the methods described herein are especially suited for remineralizing an interproximal tooth surface, i.e., between adjacent teeth.

### Kits

In many embodiments, a kit is provided. The kit may include a dental article including a at least one applicator and a silver-fluoride composition. Alternatively, the kit may include at least one applicator and a silver-fluoride composition. The silver fluoride composition(s) may include a source of silver cations, a source of fluoride anions, a source of iodide or thiocyanate anions, and water. The kit may further include a set of instructions direct a user perform method steps for reducing bacteria at a tooth surface described herein or methods steps for remineralizing a tooth surface described herein.

In some embodiments, the kit may include any dental article described herein.

In some embodiments, the kit may include instructions directing a user to follow any method described herein.

In many embodiments, the kit may include any applicator(s) described herein.

In many embodiments, the kit may include any silver-fluoride composition described herein, including any source of cations, anions, or the like described herein.

In many embodiments, the kit may include any package(s) described herein.

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

TABLE 1
Materials

| Description | Source | Location |
|---|---|---|
| Ammonium chloride | VWR | West Chester, PA |
| (NH₄Cl) | | |
| Ammonium iodide | Honeywell Specialty | Seelze, Germany |
| (NH₄I) | Chemical | |
| Ammonium thiocyanate | Alfa Aesar | Ward Hill, MA |
| (NH₄SCN) | | |
| Disodium hydrogen | Alfa Aesar | Ward Hill, MA |
| citrate sesquihydrate | | |
| Guanidinium thiocyanate | Alfa Aesar | Ward Hill, MA |
| Sodium carbonate | EMD | Gibbstown, NJ |
| Potassium phosphate | Sigma Aldrich | St Louis, MO |
| monobasic | | |
| Potassium sulfate | J. T. Baker | Phillipsburg, NJ |
| Silver fluoride (AgF) | Oakwood Chemical | West Columbia, SC |
| Silver iodide (AgI) | Sigma Aldrich | St Louis, MO |
| Silver diamine fluoride | Elevate Oral Care | West Palm Beach, |
| (SDF) solution | | FL |
| 38%, commercially | | |
| available as | | |
| ADVANTAGE ARREST | | |
| silver diamine | | |
| fluoride | | |
| 3M ™ ESPE ™ | 3M ESPE Company | St. Paul, MN, USA |
| Peridex ™ Chlorhexidine | | |
| Gluconate 0.12% | | |
| Oral Rinse | | |
| ORAL-B SUPER FLOSS | Proctor & Gamble | Cincinnati, OH, |
| | Company | USA |
| Aluminum foil pouch, | Oliver Healthcare | Grand Rapids, MI, |
| 10 cm × 20 cm, heat | Packaging, Inc. | USA |
| sealable; part #87891 | | |

### Example Preparation

The general sample preparation procedure was as follows. Exact percent quantities are described in the tables below. An amount of 0.5 gram of the silver compound was added to an appropriately sized plastic tube. The full amount of water (described in tables below) was added to the container to dissolve the silver compound. The remaining component(s) were added to the silver compound solution. Initially, this addition caused a precipitate to occur. For examples of the present disclosure, the continued addition of the full amount of the remaining components caused the precipitate to re-dissolve, as the one-part composition (e.g., solution) was prepared. For comparative examples, the continued addition of the full amount of the remaining components did not re-dissolve the precipitate, the precipitate remained.

TABLE 2
Examples Ex-1 to Ex-5 and Comparative Example C-8;
w/NH₄SCN; Water Less than 57%

| Component | Ex-1 | Ex-2 | Ex-3 | Ex-4 | Ex-5 | C-8 |
|---|---|---|---|---|---|---|
| AgF | 22.7 | 20.8 | 19.2 | 17.9 | 16.7 | 14.3 |
| NH₄SCN | 45.5 | 41.7 | 38.5 | 35.7 | 33.3 | 28.6 |
| H₂O | 31.8 | 37.5 | 42.3 | 46.4 | 50.0 | 57.1 |
| Total (%) | 100 | 100 | 100 | 100 | 100 | 100 |
| Ag % | 19.3 | 17.7 | 16.4 | 15.2 | 14.2 | 12.1 |
| Fluoride % | 3.4 | 3.1 | 2.9 | 2.7 | 2.5 | 2.1 |
| Ag/SCN mole ratio | 0.30 | 0.30 | 0.30 | 0.30 | 0.30 | 0.30 |
| Precipitate formed initially | Yes | Yes | Yes | Yes | Yes | Yes |
| Precipitate dissolved with | Yes | Yes | Yes | Yes | Yes | No |
| all chemicals in | | | | | | |

TABLE 3
Comparative Examples C-1 to C-7

| Components | C-1 | C-2 | C-3 | C-4 | C-5 | C-6 | C-7 |
|---|---|---|---|---|---|---|---|
| AgF | 16.7 | 16.7 | 16.7 | 16.7 | 16.7 | 18.4 | 18.5 |
| Disodium hydrogen | 50.0 | 0 | 0 | 0 | 0 | 0 | 0 |
| citrate sesquihydrate | | | | | | | |
| Sodium carbonate | 0 | 50.0 | 0 | 0 | 0 | 0 | 0 |
| Potassium phosphate | 0 | 0 | 50.0 | 0 | 0 | 0 | 0 |
| monobasic | | | | | | | |
| NH₄Cl | 0 | 0 | 0 | 50.0 | 0 | 22.1 | 0 |
| NH₄SCN | 0 | 0 | 0 | 0 | 0 | 0 | 22.2 |
| NH₄I | 0 | 0 | 0 | 0 | 0 | 22.8 | 22.2 |
| Potassium sulfate | 0 | 0 | 0 | 0 | 50.0 | 0 | 0 |
| Deionized (DI) | 33.3 | 33.3 | 33.3 | 33.3 | 33.3 | 36.8 | 37.0 |
| water | | | | | | | |
| Total (%) | 100 | 100 | 100 | 100 | 100 | 100 | 100 |
| Ag % | 14.2 | 14.2 | 14.2 | 14.2 | 14.2 | 15.6 | 15.7 |
| Fluoride % | 2.5 | 2.5 | 2.5 | 2.5 | 2.5 | 2.8 | 2.8 |
| Ag/SCN mole ratio | 0 | 0 | 0 | 0 | 0 | 0 | 0.5 |
| Ag/I mole ratio | 0 | 0 | 0 | 0 | 0 | 0.92 | 0.95 |
| Precipitate formed | Yes | Yes | Yes | Yes | Yes | Yes | Yes |
| initially | | | | | | | |
| Precipitate dissolved | No | No | No | No | No | No | No |
| with all chemicals in | | | | | | | |

TABLE 4
Examples Ex-6 to Ex-9 and Comparative
Example C-9 w/NH₄I; Water Less than 41%

| Component | Ex-6 | Ex-7 | Ex-8 | Ex-9 | C-9 |
|---|---|---|---|---|---|
| AgF | 19.2 | 17.9 | 16.7 | 15.6 | 14.7 |
| NH₄I | 57.7 | 53.6 | 50.0 | 46.9 | 44.1 |
| H₂O | 23.1 | 28.6 | 33.3 | 37.5 | 41.2 |
| Total (%) | 100 | 100 | 100 | 100 | 100 |
| Ag % | 16.4 | 15.2 | 14.2 | 13.3 | 12.5 |
| Fluoride % | 2.9 | 2.7 | 2.5 | 2.3 | 2.2 |
| Ag/I mole ratio | 0.38 | 0.38 | 0.38 | 0.38 | 0.38 |
| Precipitate formed initially | Yes | Yes | Yes | Yes | Yes |
| Precipitate dissolved with | Yes | Yes | Yes | Yes | No |
| all chemicals in | | | | | |

TABLE 5
Examples Ex-10 to Ex-12 and Comparative Example C-10;
With NH₄SCN; Silver to Thiocyanate Mole Ratio
Less than 0.37 to Dissolve Precipitate and Form a Solution

| Component | Ex-10 | Ex-11 | Ex-12 | C-10 |
|---|---|---|---|---|
| AgF | 20.0 | 21.7 | 22.7 | 23.8 |
| NH₄SCN | 40.0 | 39.1 | 45.5 | 38.1 |
| H₂O | 40.0 | 39.1 | 31.8 | 38.1 |
| Total (%) | 100 | 100 | 100 | 100 |
| Ag/SCN mole ratio | 0.30 | 0.33 | 0.30 | 0.37 |
| Ag % | 17.0 | 18.5 | 19.3 | 20.2 |
| Fluoride % | 3.0 | 3.3 | 3.4 | 3.6 |
| Precipitate formed initially | Yes | Yes | Yes | Yes |
| Precipitate dissolved with | Yes | Yes | Yes | No |
| all chemicals in | | | | |

TABLE 6
Example Ex-13 and Comparative Examples C-11 to C-13;
With NH₄I; Silver to Iodide Mole Ratio Less than 0.42
to Dissolve the Precipitate and Form a Solution

| Component | Ex-13 | C-11 | C-12 | C-13 |
|---|---|---|---|---|
| AgF | 17.2 | 18.2 | 19.2 | 20.8 |
| NH₄I | 51.7 | 49.1 | 46.2 | 41.7 |
| H₂O | 31.0 | 32.7 | 34.6 | 37.5 |
| Total (%) | 100 | 100 | 100 | 100 |
| Ag/Iodide mole ratio | 0.38 | 0.42 | 0.48 | 0.57 |
| Ag % | 14.7 | 15.5 | 16.4 | 17.7 |
| Fluoride % | 2.6 | 2.7 | 2.9 | 3.1 |
| Precipitate formed initially | Yes | Yes | Yes | Yes |
| Precipitate dissolved with | Yes | No | No | No |
| all chemicals in | | | | |

TABLE 7
Examples Ex-14 and Ex-15

| Component | Ex-14 | Ex-15 |
|---|---|---|
| AgI | 31.0 | 0 |
| AgF | 0 | 18.5 |
| NH₄I | 31.4 | 0 |
| NH₄F | 4.9 | 0 |
| Guanidinium thiocyanate | 0 | 51.9 |
| DI water | 32.7 | 29.6 |
| Total % | 100 | 100 |
| Ag % | 14.3 | 15.7 |
| Total Iodine % | 44.4 | 0 |
| Total Fluoride % | 2.5 | 2.8 |
| Precipitate formed initially | Yes | Yes |
| Precipitate dissolved with all chemicals in | Yes | Yes |

### Example 16

Silver diamine fluoride water solution (38%) (commercially available as ADVANTAGE ARREST from Elevate Oral Care of West Palm Beach, Fla., USA) was used as source of silver and fluoride. An amount of 0.1 gram of silver diamine fluoride solution was mixed with 0.248 gram of NH₄I, the mixture released ammonia and a little bit of NH₄I did not dissolve and a precipitate was initially formed. The mixture then became a clear solution. This example demonstrated that silver diamine fluoride solution can be used as the source of silver and fluoride. The SDF solution was then converted to the disclosed composition of this disclosure by the addition of the appropriate amount of counter ion, NH₄I.

### Light Sensitivity Testing of Examples C-14, Ex-17, Ex-18, Ex-19

The following examples demonstrated that solutions of the disclosed composition do not turn (discolor) to black or grey after (1) being precipitated with the addition of a buffer solution (to mimic saliva in the oral environment), and (2) exposure to light using 3M ELIPAR DEEPCURE-S LED curing light with wavelength around 450 nm and output approximately 1500 mW/cm² for 20 seconds.

### Comparative Example 14 (C-14)

An amount of 25 mg of silver diamine fluoride solution (ADVANTAGE ARREST silver diamine fluoride solution (38%)) was mixed with 40 mg of 1% KH₂PO₄ water solution. The mixture formed a precipitate. The mixture was exposed to a blue LED light using 3M ELIPAR DEEPCURE-S LED curing light, with wavelength around 450 nm and output approximately 1500 mW/cm² for 20 seconds, the mixture turned black.

### Example 17

An amount of 23 mg of Ex-4 solution was mixed with 42 mg of 1% KH₂PO₄ water solution. The mixture formed a precipitate. The mixture was exposed to a blue LED light using 3M ELIPAR DEEPCURE-S LED curing light, with wavelength around 450 nm and output approximately 1500 mW/cm² for 20 seconds, the mixture did NOT turn (discolor) to black or grey.

### Example 18

An amount of 23 mg of Ex-7 solution was mixed with 43 mg of 1% KH₂PO₄ water solution. The mixture formed a precipitate. The mixture was exposed to a blue LED light using 3M ELIPAR DEEPCURE-S LED curing light, with wavelength around 450 nm and output approximately 1500 mW/cm² for 20 seconds, the mixture did NOT turn (discolor) to black or grey.

### Example 19

An amount of 48 mg of Ex-16 solution was mixed with 100 mg of 1% KH₂PO₄ water solution. The mixture formed a precipitate. The mixture was exposed to a blue LED light using 3M ELIPAR DEEPCURE-S LED curing light, with wavelength around 450 nm and output approximately 1500 mW/cm² for 20 seconds, the mixture did NOT turn (discolor) to black or grey.

### Solution Stability Testing

Example 14 (Ex-14) was used to assess the long-term solution stability of the solution of the present disclosure. In each sample 1 mL of Example 14 solution was placed in a plastic test tube, which was sealed in a glass vial to prevent evaporation. The samples were stored for several months under the temperature conditions described below and tested periodically at designated time points. Each sample was tested for General Appearance, Precipitation with KH₂PO₄, and Color Change (Black staining). Additional select samples were also tested for fluoride content over time. The test results show that the representative example showed no sign of unacceptable appearance and maintained acceptable levels of deliverable fluoride content over the test stability storage intervals.

### Appearance Testing

Appearance was tested by visually observing any precipitation formed in the solution (naturally) or color change during aging/stability study.

### Precipitation Testing

A precipitation test was performed by inducing precipitation by the introduction of one drop of 1% monopotassium phosphate (KH₂PO₄) and observing any formed precipitate.

### Color Change (Staining) Testing

Color change was performed by exposing the precipitate formed with the addition of KH₂PO₄ to blue LED light using 3M ELIPAR DEEPCURE-S LED curing light, with wavelength around 450 nm and output approximately 1500 mW/cm2 for 20 seconds. This test was performed to look for dark (e.g., black, brown, or grey) discoloration of the formed precipitate, which is an undesirable characteristic of the discoloration of silver diamine fluoride+KH₂PO₄ precipitate when exposed to light. Examples of the present disclosure do not exhibit this undesirable light sensitivity characteristic.

### Fluoride Content Testing

Fluoride level in parts per million (ppm) was measured for select samples using a Mettler Toledo T70 titrator. The Cole Parmer fluoride electrode was first calibrated with parts per million (ppm) fluoride standards with TISAB III before measuring samples for fluoride content. Total Ionic Strength Adjustment Buffer (TISAB) III concentrate solution is for use with fluoride ion selective electrodes and is available from Sigma Aldrich. The fluoride ion selective electrode was placed in the titrator cup of diluted TISAB III solution and allowed to equilibrate for 30 seconds before analyzing each sample. The fluoride content in ppm was calculated against the fluoride standards calibration curve.

TABLE 11
Stability Testing at Room Temperature (25° Celsius) of Example 14

| Time | | Precipitation | Color Change/ |
|---|---|---|---|
| (Month) | Appearance | after KH₂PO₄ | Black Stain |
| 0 | Clear, faint yellow solution; | Yes | No |
| | no undissolved particles. | | |
| 1 | Same as time 0. | Yes | No |
| 3 | Same as time 0. | Yes | No |
| 6 | Same as time 0. | Yes | No |
| 9 | Clear, slightly darker yellow | Yes | No |
| | than time 0; no undissolved | | |
| | particles. | | |

TABLE 12
Stability Testing at 37° Celsius of Example 14

| Time | | Precipitation | Color Change/ |
|---|---|---|---|
| (Month) | Appearance | after KH₂PO₄ | Black Stain |
| 0 | Clear, faint yellow solution; | Yes | No |
| | no undissolved particles. | | |
| 1 | Same as time 0. | Yes | No |
| 3 | Same as time 0. | Yes | No |
| 6 | Same as time 0. | Yes | No |
| 9 | Clear, slightly darker yellow | Yes | No |
| | than time 0; no undissolved | | |
| | particles. | | |

TABLE 13
Stability Testing at 45° Celsius of Example 14

| Time | | Precipitation | Color Change/ | Total Fluoride |
|---|---|---|---|---|
| (Month) | Appearance | after KH₂PO₄ | Black Stain | (ppm) |
| 0 | Clear, faint yellow | Yes | No | 25799 |
| | solution; no undissolved | | | |
| | particles. | | | |
| 1 | Same as time 0. | Yes | No | Not tested |
| 2 | Same as time 0. | Yes | No | Not tested |
| 3 | Same as time 0. | Yes | No | Not tested |
| 6 | Clear, slightly darker | Yes | No | 27359 |
| | yellow than time 0; no | | | |
| | undissolved particles | | | |
| 9 | Same as 6 months. | Yes | No | Not tested |

TABLE 14
Stability Testing at 60° Celsius of Example 14

| Time | | Precipitation | Color Change/ | Total Fluoride |
|---|---|---|---|---|
| (Month) | Appearance | after KH₂PO₄ | Black Stain | (ppm) |
| 0 | Clear, faint yellow | Yes | No | 25799 |
| | solution; no undissolved | | | |
| | particles. | | | |
| 0.5 | Same as time 0. | Yes | No | Not tested |
| (2 weeks) | | | | |
| 1 | Same as time 0. | Yes | No | 25759 |
| 1.5 | Same as time 0. | Yes | No | Not tested |
| (6 weeks) | | | | |
| 2 | Clear, slightly darker | Yes | No | Not tested |
| | yellow than time 0; no | | | |
| | undissolved particles | | | |
| 2.5 | Same as 2 months. | Yes | No | Not tested |
| (10 weeks) | | | | |
| 3 | Same as 2 months. | Yes | No | 27034 |

### Antimicrobial Testing—Zone of Inhibition

A multispecies zone of inhibition test was performed according to the following steps in order to assess antimicrobial efficacy of comparative examples and Example 14 (Ex-14) as a representative of the present disclosure. Human saliva was used as the source of bacteria.
1. Sterilized 6 mm diameter round filter paper disks were used.
2. Experimental and control solutions were dispensed into standard 96 well plates in preparation to soak the filter paper disks.
3. Each sterilized paper disk was soaked by applying 10 μL of corresponding solution to the disks. Each sample was done in triplicate and final results were averaged.
4. Human saliva and mucin containing medium mixture was spread on the agar plate. Agar plates were divided into equal sections according to the number of solutions to be tested. Sections were marked with the names of the test solutions.
5. The agar plate lid was lifted off and the soaked filter paper samples were placed onto the agar. Each sample filter paper disk was then gently pushed down to ensure complete contact of the filter paper disk with the agar surface. Caution was taken to not move a disk once it contacted the agar surface.
6. The analyst continued to place one sample disk at a time onto the agar surface until all disks were placed into their respective sections of each agar plate, as described above.
7. Once all disks were in place, the lid was replaced, the agar plates were inverted, and placed in a 37° C. air incubator for 24 hours.
8. After 24 hours incubation at 37° C., the diameter of each zone of inhibition was measured to the nearest millimeter using a ruler or calipers. The zones of inhibition were measured as the diameter from the edges of the last visible colony, according to the unaided eye.
9. During measurements, care was taken to not touch the disk or surface of the agar. The ruler was decontaminated between each measurement; measurements were performed in a biosafety cabinet.

TABLE 15
Zone of Inhibition Antimicrobial Testing Results

| | Zone of Inhibition | Std. Dev. |
|---|---|---|
| Sample | Diameter Ave. (mm) | (n = 3) |
| 0.9% saline | 6 | 0 |
| PERIDEX CHG 0.12% Oral Rinse | 12 | 0 |
| ADVANTAGE ARREST 38% SDF | 13 | 0 |
| Ex-14 | 13 | 0 |

### Remineralization and Acid Resistance Testing

Specimens of enamel were prepared from bovine teeth. Caries lesions were created in the enamel by immersing each specimen in a solution of 0.1M lactic acid and 0.2% CARBOPOL (pH=5) at 37° C. for 24 hours. The specimens were randomized to one of the following treatment liquids: artificial saliva (used as a control), silver diamine fluoride (SDF) as a comparative solution, Example 14 (Ex-14). A total of 10 specimens per treatment solution were prepared.

The specimens were treated by applying the different test treatment liquids on the enamel lesion with a mini dental bush, rubbing the lesion with the mini dental brush for 10 seconds, waiting for one minute, rinsing with artificial saliva, and storing in artificial saliva for 30 minutes at 37° C. Specimens were then rinsed with artificial saliva again and placed in fresh artificial saliva for 24 hours at 37° C.

The surface hardness (Vickers Hardness Number (VHN)) of treated specimens was measured by a Vickers hardness tool. The average VHN and standard deviation of the 10 specimens per treatment were calculated.

To measure the retention of the surface micro-hardness, the specimens were next placed in a solution of 0.1M lactic acid and 0.2% CARBOPOL (pH=5) for 24 hours to create a demineralization challenge. The retention of any increased hardness was evaluated via acid challenge. Samples were subjected to demineralization solution for 24 hours. Retention of micro-hardness following the acid challenge indicates an ability of the treatment to resist demineralization. Surface micro-hardness was measured following the acid challenge. Micro-hardness values that were higher than baseline measurements indicated remineralization of tooth enamel by the treatment. The specimens were stored in artificial saliva for 24 hours at 37° C., and then hardness was measured. The specimens were acid challenged by demineralization solution and the hardness were measured again. The more resistant the enamel to the challenge, the higher the retention of the surface micro-hardness value. Surface micro-hardness was measured using the same procedure as with the baseline measurement. Any increase in micro-hardness after treatment indicates an ability of the treatment solution to remineralize tooth enamel.

TABLE 16
Enamel Micro-hardness (VHN) and Remineralization Testing Results

| | Saliva | | |
|---|---|---|---|
| Treatment | Control | SDF | Ex-14 |
| Baseline Average | 50.83 | 52.39 | 53.12 |
| Baseline Std. Dev. | 12.55 | 12.40 | 13.05 |
| Treatment Average | 77.52 | 96.31 | 84.75 |
| Treatment Std. Dev. | 16.02 | 18.04 | 21.33 |
| Acid Challenge Average | 41.32 | 74.04 | 84.37 |
| Acid Challenge Std. Dev. | 9.24 | 10.21 | 22.46 |
| Saliva Remin. Average | 54.21 | 105.84 | 108.08 |
| Saliva Remin. Std. Dev. | 10.66 | 16.06 | 25.52 |
| Second Acid Challenge Average | 29.16 | 70.86 | 85.75 |
| Second Acid Challenge Std. Dev. | 6.91 | 13.60 | 19.64 |

### Fluoride Uptake Testing

Specimens of enamel were prepared from bovine teeth. Caries lesions were created in the enamel by immersing each specimen in a solution of 0.1M lactic acid and 0.2% CARBOPOL (pH=5) at 37° C. for 24 hours. The specimens were randomized to one of the following treatment liquids: artificial saliva (used as a control), silver diamine fluoride (SDF) used as a comparison, Example 14 (Ex-14). A total of 10 specimens per treatment group were prepared.

The specimens were treated by applying the different test treatment liquids on the enamel lesion with a mini dental bush, rubbing the lesion with the mini dental brush for 10 seconds, waiting for one minute, rinsing with artificial saliva, and storing in artificial saliva for 30 minutes at 37° C. Specimens were then rinsed with artificial saliva again and placed in fresh artificial saliva for 24 hours at 37° C.

Microdrill biopsies were taken of each specimen to measure the amount of fluoride transferred to the enamel. To test the fluoride incorporation into the tooth, specimens were further subjected to an acid challenge. Specimens were placed into a solution of 0.1M lactic acid and 0.2% CARBOPOL (pH=5) at 37° C. for 24 hours to simulate an acid challenge. Microdrill biopsies were taken from each specimen to measure the amount of fluoride in the enamel following acid challenge. Results in units of μg F/cm² are reported in Table 17.

TABLE 17
Fluoride Uptake (μg F/cm²) After Example Solution Treatments

| | | | Acid | |
|---|---|---|---|---|
| | Treatment | | Challenge | Acid |
| | Average | Treatment | Average | Challenge |
| Treatment | (μg F/cm²) | Std. Dev. | (μg F/cm²) | Std. Dev. |
| Saliva control | 2.64 | 1.15 | 3.66 | 1.97 |
| SDF | 20.81 | 4.50 | 20.92 | 6.01 |
| Ex-14 | 36.69 | 9.12 | 27.00 | 10.87 |

### Accelerated Weight Loss Testing in Foil Pouch

Example 20 (Ex-20) was prepared in the following manner. The selected applicator was commercially available ORAL-B SUPER FLOSS, which has a stiffened-end dental floss threader portion, a spongy floss portion, and a regular floss portion. The spongy floss portion of the ORAL-B SUPER FLOSS acts as an absorbent portion of the applicator. An amount of Example 14 (Ex-14) was prepared. The spongy floss portion of the ORAL-B SUPER FLOSS was dipped into the solution of Example 14 to absorb an amount of Example 14. The entire ORAL-B SUPER FLOSS, soaked with Example 14, was placed in an aluminum foil pouch (Oliver Pouch, part #87891, 10 cm 20 cm; commercially available from Oliver Healthcare Packaging, Inc.). The foil pouch was sealed with a hand-pressed Impulse sealer, model type A1E-300, with 300 watts AC120V 60 Hz, available from American International Electric Sealer Supply. Example 20 represents a single use package of one embodiment of the present disclosure.

Replicates (A-E) of Example 20 were prepared and stored at 60° C. for 1-month and 2-month intervals. The percent weight loss over those intervals were calculated and found to be acceptable. Storage at 60° for 2 months is generally considered to be equivalent to 2 years storage at room temperature.

TABLE 18
Accelerated Weight Loss Testing in Foil Pouch for 1 month at 60° C.

| | Time 0 | Time 0 | Time 0 | Time 0 | | Wt. Diff. | % Diff |
|---|---|---|---|---|---|---|---|
| | SUPER | pouch | Amount | Total | 1 Month | Time 0 wt. | Percent |
| | FLOSS | wt. | Ex-14 | wt. | Total wt. | minus | Diff. in |
| Example | (grams) | (grams) | (grams) | (grams) | (grams) | 1-month wt. | weight |
| Ex-20-A | 0.0655 | 2.331 | 0.1860 | 2.5827 | 2.5866 | −0.004 | −0.2% |
| Ex-20-B | 0.0666 | 2.331 | 0.2294 | 2.6273 | 2.5791 | 0.048 | 1.8% |
| Ex-20-C | 0.0653 | 2.342 | 0.2104 | 2.6173 | 2.6215 | −0.004 | −0.2% |

| | |
|---|---|
| Average % weight change over 1 month at 60° C. in foil pouch | 0.5% |

TABLE 19
Accelerated Weight Loss Testing in Foil Pouch for 2 months at 60° C.

| | Time 0 | Time 0 | Time 0 | | | Wt. Diff | % Diff |
|---|---|---|---|---|---|---|---|
| | SUPER | pouch | Amount | Time 0 | 2 Months | Time 0 wt. | Percent |
| | FLOSS | wt. | Ex-14 | Total wt. | Total wt. | minus | Diff- in |
| Example | (grams) | (grams) | (grams) | (grams) | (grams) | 2-month wt. | weight |
| Ex-20-D | 0.0610 | 2.294 | 0.197 | 2.5522 | 2.5571 | −0.005 | −0.2% |
| Ex-20-E | 0.0655 | 2.331 | 0.1978 | 2.5946 | 2.5999 | −0.005 | −0.2% |

| | |
|---|---|
| Average % weight change over 2 months at 60° C. in foil pouch | −0.2% |

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A dental article configured to access an interproximal gap, the dental article comprising:
at least one applicator;
a silver-fluoride composition comprising:
a source of silver cations,
a source of fluoride anions,
a source of iodide or thiocyanate anions, and
water; and
a package for containing one or more of the at least one applicator and the silver-fluoride composition,
wherein the package is impermeable to water vapor.

2. (canceled)

3. The dental article of claim 1, wherein the applicator is selected from a floss, a floss pick, a pick, a microbrush, a swab, or a combination thereof.

4. The dental article of claim 1, wherein the applicator is pre-saturated with the silver-fluoride composition.

5. The dental article of claim 1, wherein silver cations are present in an amount of 13-20 wt % with respect to the weight of the silver-fluoride composition.

6. The dental article of claim 1, wherein the fluoride anions are present in an amount of 2.0-3.6 wt % with respect to the weight of the silver-fluoride composition.

7. The dental article of claim 1, comprising:
silver cations and iodide anions in a molar ratio of less than 0.42:1, and
water present in an amount less than 41.2 wt % with respect to the weight of the dental composition.

8. The dental article of claim 1, comprising:
silver cations and thiocyanate anions in a molar ratio of less than 0.37:1, and
water present in an amount less than 57.1 wt % with respect to the weight of the dental composition.

9. The dental article of claim 1, wherein water is present in an amount less than about 45 wt % with respect to the weight of the silver-fluoride composition.

10. The dental article of claim 1, wherein water is present in an amount of at least about 20 wt % with respect to the weight of the silver-fluoride composition.

11. The dental article of claim 1, wherein the source of silver cations is selected from silver fluoride, silver chloride, silver nitrate, silver iodide, silver diamine fluoride, and a combination thereof.

12. The dental article of claim 1, wherein the source of fluoride anions is selected from silver fluoride, silver diamine fluoride, sodium fluoride, ammonium fluoride, potassium fluoride, an organic ammonium fluoride, and a combination thereof.

13. The dental article of claim 1, wherein the source of iodide anions is selected from ammonium iodide, sodium iodide, potassium iodide, silver iodide, and a combination thereof, or
the source of thiocyanate anions is selected from ammonium thiocyanate, sodium thiocyanate, guanidinium thiocyanate, potassium thiocyanate, silver thiocyanate, and a combination thereof.

14. The dental article of claim 1, the silver-fluoride composition comprising silver iodide, ammonium iodide, and ammonium fluoride.

15. (canceled)

16. A method for one or more of reducing bacteria on a tooth surface and remineralizing a tooth surface, the method comprising:
providing a dental article of claim 1;
contacting an effective amount of the silver-fluoride composition to the tooth surface with the at least one applicator; and
allowing the silver-fluoride composition to contact the tooth surface for a period,
wherein the silver-fluoride composition one or more of reduces the bacteria at the tooth surface and remineralizes the tooth surface.

17. The method of claim 16, further comprising contacting the at least one applicator to the tooth surface and loading the applicator with the silver-fluoride composition.

18. The method of claim 16, wherein the at least one applicator is pre-loaded with the silver-fluoride composition.

19. The method of claim 16, wherein the silver-fluoride composition to allowed to contact the tooth surface for a period of about 60 to about 90 seconds.

20. The method of claim 16, wherein the effective amount is at least about 30 mg of the silver-fluoride composition.

21. A method of claim 16, wherein the tooth surface is an interproximal tooth surface.

22. A kit comprising:
a dental article of claim 1, or
at least one applicator, and
a silver-fluoride composition comprising:
a source of silver cations,
a source of fluoride anions,
a source of iodide or thiocyanate anions, and
water; and
a set of instructions directing a user to contact an effective amount of the silver-fluoride composition to the tooth surface with the at least one applicator.
